(19) United States
(12) Patent Application Publication
(10) Pub. No.: US 2012/0330127 A1
(43) Pub. Date: Dec. 27, 2012

(54) GENERATION OF SCAN DATA AND FOLLOW-UP CONTROL COMMANDS

(76) Inventor: PETER AULBACH, Forchheim-Kersbach (DE)

(21) Appl. No.: 13/526,643

(22) Filed: Jun. 19, 2012

(30) Foreign Application Priority Data
Jun. 24, 2011 (DE) 10 2011 078 039.4

(51) Int. Cl. A61B 6/00

(52) U.S. Cl. 600/407

(57) ABSTRACT

A method to generate scan data with a medical imaging technology system includes the steps of receiving control commands to control an imaging scan process and/or an image output via a user interface, implementing an imaging scan process with the imaging system, providing image data generated by the scan process; and linking the image data with a data set representing the user interface during the reception to form the scan data. A medical imaging technology system is designed to implement such a method.

### BACKGROUND OF THE INVENTION

1. Field of the Invention

The present invention concerns a method to generate scan data by means of a medical imaging technology system. It moreover concerns a medical imaging technology system.

2. Description of the Prior Art

Generally included under “medical imaging technology systems” are all medical technology systems that can reveal the inside of bodies entirely or in parts, in particular living bodies, primarily animals or people, by means of imaging methods. Computed tomography (CT) systems, magnetic resonance (MR) systems, angiography systems, ultrasound apparatuses and x-ray apparatuses are examples.

In recent years, significant advances have been under in medical imaging technology, both with regard to the control of the actual scan process (i.e. the image acquisition) and with regard to the image processing and output. At present, different radiation dose reductions (in CT, for instance) can be activated, partially activated, deactivated or modified in the preparation of tomography scans, for example. This manner of operation is known as active dose management. Now it can be simulated how the dose would be distributed across a patient body in computed tomography scans before the scanner itself is activated. Via a graphical user interface, the overview of all available dose reduction possibilities can be displayed in advance. It is thereupon apparent which possibilities for dose reduction present in the specifically planned scan and which are actually provided for this scan.

Furthermore, within the scope of tomographic imaging it is now possible to plan and guide minimally invasive procedures. Such minimally invasive procedures are endoscopy procedures to ablate tissue, to suction away fluids or the like, for example. To plan such procedures, with the use of current scan image data, a procedure planning can take place that establishes a (direct or multi-directional) path from a puncture location of a procedure instrument to the actual point of action in the body, which path is then traversed during the actual procedure.

During such a minimally invasive procedure, additional image data can be generated in parallel by the medical imaging technology system so that the location of the procedure instrument can be tracked at any time and follow-up corrections by a treating physician can be made if necessary.

The presentation of the image data that were generated by an imaging tomographic scan takes place via a graphical user interface, such as a screen display in connection with an input device (a mouse, for example) and/or an output device such as a printer. The image data are furthermore often stored in a standardized format according to the DICOM standard.

The time at which the patient was exposed to scanning radiation, and the scan dose of the radiation and the body region that was scanned are shown to a user from such DICOM image data. In the final effect, a user thus has only limited data at hand that roughly indicate the circumstances under which a scan has taken place and who the respective patient is.

Neither the DICOM standard nor its extension in what is known as the presently projected DICOM Structured Report (DSR) offer the possibility to make the control of the scan process (and, as a result, also of the image preparation) retraceable so that this satisfies modern standards of quality control for processes.

However, the controls by the user are decisively relevant to quality in multiple ways. First, in the scan planning the user (meaning the operator of the imaging system) determines the type, quality and significance of the image data resulting from the scan. Second, the user can expose the patient (or in general the examination subject) with higher or lower doses of radiation, waves, or magnetic fields, and contrast agent if necessary. Specifically in the case of patients who are frequently exposed to a similarly developed dose (for example those having chronic illnesses) or in the case of particularly sensitive patients such as children, care to be taken to ensure that the dose is made to be as low as possible. Third, the significance of the image data is also dependent on how a user prepares the image data for presentation. Items of interest in a tissue can thus be detected or lost depending on the perspective, the manner of the slice presentation (slice depth, for example) and many other factors.

In addition to the quality aspect, another factor is that the control by a user should also be reproducible for scientific and/or training purposes.

### SUMMARY OF THE INVENTION

An object of the present invention is to provide for optimally comprehensive and/or significant reproducibility of the control of such an imaging system.

This object is achieved in accordance with the invention by a method of the aforementioned general type that includes the following steps that (in principle) can take place in an arbitrary order, but preferably in the order in which they are listed.

Control commands to control an imaging scan process and/or an image output are received via a user interface. An imaging scan process is implemented by the imaging system. Image data generated by the scan process are linked with a data set representing the user interface during the reception to form the scan data.

The user interface that is used in the course of the method is preferably an integral component of the imaging system, but it can also be an external unit linked with the imaging system or comprise units that are a component of the imaging system, and other units that are to be viewed as external to the imaging system. A particularly suitable user interface is a graphical user interface, i.e. a graphical user interface with integrated or connected input device (a computer mouse, for instance), but the invention is not limited to this. For example, a speech input interface—a dictation system, for example—can also act as a user interface via which control commands are relayed to the imaging system.

In general, both control commands, such as clicking on a start button to begin a defined process, and parameter value inputs to determine the framework data of a process, are defined as control commands, as well as control data based on an image. For example, plans for follow-up scans and/or for procedures (minimally invasive procedures, for example) can be integrated into graphical renderings image data (for instance from an overview scan). These are likewise to be considered as control commands within the framework of the scan process or, respectively, the image post-processing, as well as any other control data that directly relate to the image acquisition or, respectively, image output.

The control commands preferably are those control commands that are modified in comparison to control commands predetermined in a scan protocol of the imaging system. This means that at least one predetermined control command from the scan protocol is adapted according to the desires of the user upon receiving control commands. It is precisely in the modification of predetermined control commands that it is particularly important to document what the alterations are that the user has entered. For example, in the sense of quality assurance a reasonable (i.e. in particular effective) tracking of critical work steps can hereby be achieved.

The provision of image data can be the storage of image data and/or the display (for example by means of a screen and/or via a printer) of the image data for a user. A data set is now consequently assigned to these image data or is combined with the image data. This takes place within the framework of a link that is to be understood as analogous to a hyperlink in a conventional personal computer system, and can also be (but does not necessarily need to be) designed similarly in terms of the logic. For example, the image data and the data set can simply be stored with one another in a common data folder, or the data set can be added as a type of image into the image data in the image. Within the framework of the invention, it is imperative for the link that image data and a data set are unambiguously associated with one another so that the corresponding data set can also optimally be accessed (optimally automatically) in the follow-up upon retrieving the image data. It is particularly important that a separation of the image data and the data set cannot be implemented without detectable traces or cannot be implemented at all, just like a subsequent modification of the data set.

Data known as scan data are created by the combination of the image data with the data set. In order to be able to understand the value of such scan data, the data set and its content are noted again: the data set represents the user interface in a situation during the input, i.e. during the reception of the control commands. This means that at least selected control commands are automatically included in the data set, be they in coded form or in the form of machine-readable data. The fact of the representation of the user interface additionally exists in that it is ensured that the control commands are learned directly from the user interface. No control commands are thus adopted from other data systems (for instance those that are still manipulable after the fact), but rather only those control commands that the user could learn at least from the user interface, preferably that he has modified and/or entered and/or actively confirmed himself. The data set thus also includes at least those control commands that the user has himself actively edited or confirmed.

The direct connection between the user interface (an input mask, for instance) and the received control command also preferably arises from the data set. Moreover, it is preferred that the data set comprises those control commands that are recorded in the data set within a defined time frame, preferably within ten minutes after receiving the control command, particularly preferably five minutes and very particularly preferably within one minute.

As mentioned above, those control commands are detected that have been modified with respect to the predetermined commands. Because the data set represents the user interface (or at least a portion thereof) the steps that were implemented by a user in the input procedure, or even which inputs were omitted, can be documented with the use of the data set. It is therefore important that the data set does not include just any control commands, but rather that its content represents the user interface during the reception of the control commands. In other words, a true depiction of the user interface is thus created.

Moreover, it is preferred that the scan data include control commands to control an imaging scan process. First of all, due to the DICOM standard, data of a certain depth are already present with regard to the image processing following the scan process, even if it is far from as deep as is possible within the scope of the invention. Second, the control of the scan process is especially relevant to the image quality because the raw data are provided that are then only post-processed or visualized in the image data preparation, (but may also be processed again differently as desired). Moreover, an incorrect or suboptimal control of the scan process can also have direct effects on a patient (or an examination subject in general) since radiation, wave or magnetic field doses are monitored that could lead to damage upon overdoses (also across multiple scans). In order to be able to demonstrate that the dose was optimized for the scan process, it is therefore of importance to also document the control commands to control an imaging scan process in particular.

Because the data set in the scan data is linked with the image data, the image data are connected with information that optimally reproduces the default settings of the imaging scan process and/or the image presentation without gaps. In contrast to the information that is typically stored within a DICOM image, by using the data set representing the user interface it is possible to bring a large number of (if not all) control commands that were established in advance to control the imaging system into a fixed connection with the image data.

Therefore, it is possible for the first time to ensure a gapless quality verification via input commands in medical imaging technology systems. Ultimately, the relevant control commands, or their entry are/is directly connected with the result of the tomography scan that is based on these control commands. Nevertheless, such a procedure can be implemented without larger additional effort and requires only slightly more computer capacity than before. A large synergy effect thus arises by the combination of the different data given a simultaneously low cost for the provision.

The invention also encompasses a method to generate follow-up commands for an imaging scan process via a medical imaging technology system that includes the following steps of receiving scan data generated by means of a method of the type just described according to the invention, identifying at least one control command from the data set of the scan data, and deriving at least one follow-up control command from the identified control command.

Scan data as they were initially derived in the method described above are thus consequently used to filter a number of control commands out of the data set of the scan data and to generate follow-up control commands from these. In other words: the previously generated control commands are automatically reestablished at least in part. It is thereby preferred to initially adopt the control commands from the data set and to possibly modify these only as needed. This means that a follow-up control command can also be identical to the control commands from the data set.

This method allows parameter regulations of the control commands that are made once to be used again (which was previously practically impossible) as soon as a user of the imaging system has made his own parameter value adjustments to the control commands. An automatic reestablishment of these previously modified control commands was not possibly only because the corresponding control commands no longer existed. A reestablishment of the control commands could thus be made only on the basis of handwritten notes or recollections. The method according to the invention for the generation of follow-up control commands accordingly enables a significantly increased, or even a complete, integration of control commands in successive scan processes (also multiple such processes in series), even if these are implemented at larger time intervals from one another. Because the image data are hard-linked with the data set in the scan data, the data set can be accessed again at any time, and corresponding relevant control commands can be extracted, re-used and/or modified. A significant additional benefit also results here (that can, however, be produced without noteworthy additional computational demand) that exists not only in the simplification of processes but also and in particular in the integration of medical technology standards that are set only once.

According to the invention, a medical imaging technology system of the aforementioned type has a user interface to receive control commands to control an imaging scan process and/or an image output, a scanner arrangement to implement the imaging scan process, a preparation unit that prepares the image data generated via the scan process, and a linking unit that, during operation, links the image data with a data set representing the user interface during the reception thereof to form the scan data.

The scanner arrangement is designed so that it converts the control commands received via the user interface during the scan process. It is therefore directly or indirectly connected with the user interface.

In addition to the preparation unit and/or as part of the preparation unit, the medical imaging technology system can also comprise a data set preparation module that is designed so that it automatically and/or semi-automatically produces or prepares the data set representing the user interface during the reception thereof.

All noted elements of the medical imaging technology system can be realized both in hardware and in the form of software modules and/or from hardware/software combinations. For example, every unit individually, combinations of these or all units can be realized as program modules that are executed by one or more processors.

The present invention also encompasses a non-transitory, computer-readable data storage medium encoded with programming instructions that, when the storage medium is loaded into or run on a computer, cause the computer to execute any or all of the above-described embodiments of the method.

According to a first variant of the invention, the data set represents images (or a single image). The input control commands can be at least partially derived from the graphical rendering of the respective images, meaning that the corresponding images are designed so that such a derivation (for example a readout) of control commands is possible. In other words: the images must include image regions from which control commands directly or indirectly arise. The images are preferably provided in a compatible format or in the same format as the image data (for example thus as DICOM images) so that the data set with the images and the image data can be combined with one another in a simplified manner. A snapshot of a graphical user interface is particularly suitable as an image within the scope of the invention. Analogous to a screenshot at a personal computer, one acquisition or multiple acquisitions of the user interface can thus be made from which the control commands input at the point in time of the snapshot arise. Such a snapshot or a collection of such snapshots is therefore one of the simplest possibilities of documenting the user inputs via the user interface to control the scan process or the image output.

The images can also be an image workflow sequence, meaning multiple image exposures that are correlated with one another in terms of their content and that were preferably generated at regular time intervals from one another. An image workflow sequence can also include individual images that are respectively generated automatically or semi-automatically after a defined user input. In the ideal case, each modification of a control command is individually documented by an image during the planning of the scan process or the image rendering.

According to a second variant (that is to be viewed as an alternative or as a supplement to the first variant), the data set include machine-readable data. Machine-readable data are machine-readable codes, for example in the ASCII format or in the form of a text file. For example, information regarding parameter values can be included in the control commands, and/or information regarding positioning of (virtual) controllers (in particular slide controllers) on a screen of the user interface. Machine-readable data can therefore also inherently include graphical information via corresponding representative value specifications.

While it is possible in principle to proceed according to the first or second variant, it is preferable for the data set to include both images and machine-readable data. While the images are simpler and more intuitively comprehensible to a user in the post-processing of the scan process or the image preparation, it is simpler to transfer machine-readable data in a standardized manner, to store them in databases and to otherwise additionally process them electronically. Machine-readable data have a particular significance within the scope of reuse to generate follow-up control commands. Alternatively, solely images can likewise be used for the generation of the follow-up control commands: namely, with suitable readout methods (the OCR method, for example) machine-readable data that pertain to the control commands can also be extracted from the images relatively simply.

The scan data are preferably stored in a memory system, for instance in a PACS system. In this context (but also in other application fields) it is preferred to store the scan data in a standard medical technology format, in particular the DICOM format. The storage of the scan data in the memory system has the advantage that the data can be retrieved at any time, represent a secure documentation in an electronic format and also be present so as to be reusable in the sense of the generation of follow-up control commands.

It is preferable for the scan data storage to take place so that the image data and the data set remained inseparably linked with one another for a user of the imaging system with user access rights. User access rights are those access rights that are assigned by default to a competent user of the imaging system. These are thus contrasted with administrator rights or the rights of a programmer. Through this measure the user is ultimately prevented in subsequently modifying the scan data that was generated in the first place. This security measure is important especially within the scope of quality management, but also for scientific studies, since manipulations can thus be precluded, for example.

Furthermore, it is preferred that the scan data are presented as graphically prepared for a user in a graphical presentation system. The user can thus have both the image data and the data set (and the control commands included therein) displayed immediately after the image acquisition or, respectively, image preparation, and can if necessary conduct a fine tuning (for a subsequent tomography scan, for instance).

With regard to the content of the data set, it has proven to be particularly advantageous to compose this at least partially automatically on the basis of a predefined selection rule. This ensures that in particular particularly critical control commands are represented automatically in the data set, and thus that the user cannot lose sight of them. The selection rule preferably includes at least one relevance criterion that relates to the relevance of individually input control commands and/or groups of the input control commands with regard to their effects on the image data and/or on an imaging dose. The imaging dose is the dose of radiation or electromagnetic waves or the applied magnetic field that acts on the body of the examination subject (in particular a patient) locally and/or distributed over the entire body. The greater the influence of the control commands on the quality and the type of the image data or on the quality and the type of their acquisition or on the imaging dose, the more important the consideration of these control commands in the data set. Corresponding to this relevance that can be derived from these, control commands are preferably integrated into the data set when they have a comparably higher relevance than other control commands.

The content of the data set does not need to be based only on the interests from the quality management; rather, scientific and/or training purposes or necessities can also be taken into account, for example. Therefore, it has also proven to be advantageous to select the content of the data set at least partially as defined by the user. Such a user-defined selection method can take place in addition or also as an alternative to the automatic selection that has just been described. It ultimately enables an interest-based control of the data collection, which can play a significant role given specific scientific studies.

The method according to the invention in particular develops a particular effect and relevance when the imaging scan process is implemented in accompaniment to an invasive procedure in a body of an organism, and the content of the data set comprises data with regard to a planning and/or prediction of the procedure. An invasive procedure is a minimally-invasive procedure; the body of the organism is preferably a living body. The body is preferably that of an animal or a human being. The planning of the procedure can be a path planning for a procedure instrument, for example a biopsy needle. It is precisely in the case of invasive procedures (that are accompanied by a medical imaging technology system) that the planning of the procedure on the basis of the image data plays a significant role. In particular, it is possible to adapt the planning data and the image data to one another during the procedure, such that a nominal/real comparison between planning and actual selected path is already possible during the procedure (but also afterward). The result of such a procedure is in turn an improvement of the quality documentation and a greater learning effect.

### BRIEF DESCRIPTION OF THE DRAWINGS

FIG. 1 is a flowchart of an embodiment of a method according to the invention, together with an embodiment of the follow-up method according to the invention.

FIG. 2 shows a first reproduction of a graphical user interface of a user interface for use within the scope of the method according to the invention.

FIG. 3 shows a second reproduction of the same graphical user interface.

FIG. 4 shows a third reproduction of the same graphical user interface.

FIG. 5 shows a fourth reproduction of the same graphical user interface.

FIG. 6 shows a fifth reproduction of the same graphical user interface.

FIG. 7 is a schematic illustration of an embodiment of the imaging system according to the invention.

### DESCRIPTION OF THE PREFERRED EMBODIMENTS

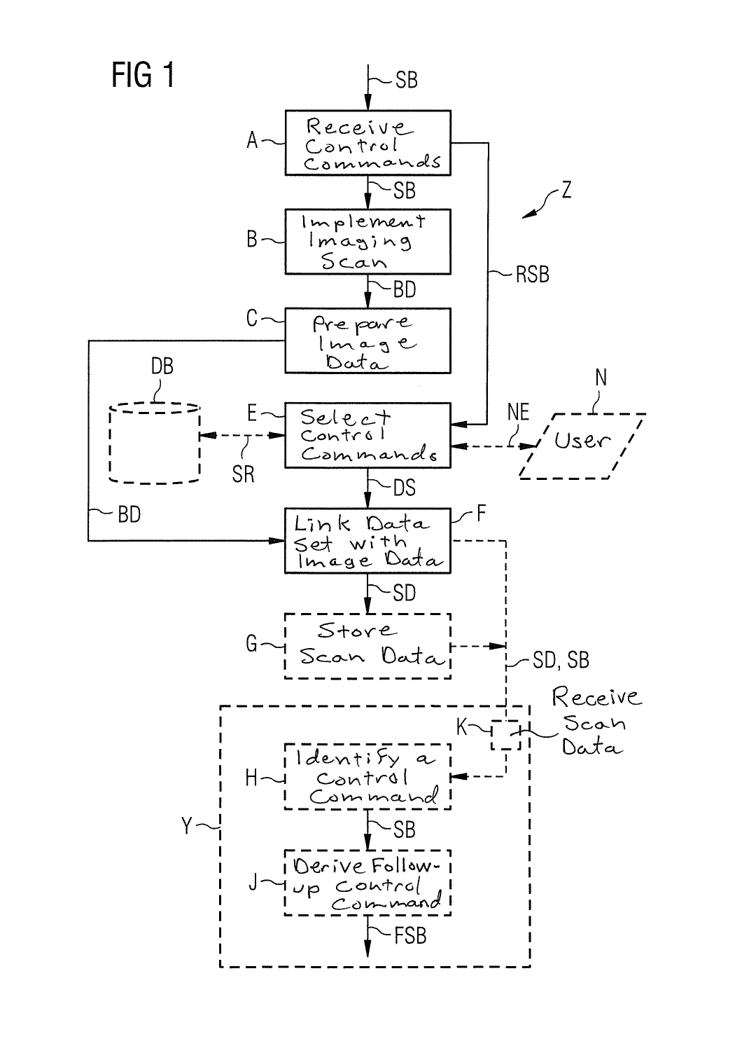

FIG. 1 is a schematic representation of the workflow of a method Z according to the invention:

In a first Step A, control commands SB are received via a graphical user interface of an imaging system. On the basis of these control commands SB, an imaging scan process is then implemented via the imaging system in a Step B. Image data BD result from this. The image data BD are prepared in Step C. A preparation of a data set DS that includes or, respectively, represents representative control commands RSB from the control commands SB takes place in parallel with this. The representative control commands RSB can include all control commands SB, or a portion of the control commands SB.

In a Step F the data set DS is linked with image data BD, meaning that they are both inseparably connected with one another for a user with conventional user rights so that they remain associated with one another, and such that in particular the data set DS is also no longer modifiable for such a user.

In Step E the representative control commands RSB can be selected on the basis of two additional inputs or comparisons for the data set DS. A selection rule SR on the basis of which specific representative control commands RSB are integrated as contents of the data set DS from a database DB as a first input. Additionally or alternatively, a user input NE by a user N can take place on the basis of which selected representative control commands RSB are likewise integrated into the data set DS. The data set DS can thereby include both images and machine-readable data, for instance in the form of text files from which the representative control commands RSB arise in encoded form or directly. Scan data SD resulting from the combination of the image data BD and the data set DS that took place in Step F, which scan data SD can be stored in an optional Step G in a memory system, for example a patient archiving system (PACS).

Moreover, the workflow of a method Y according to the invention for generation of follow-up control commands FSB is shown in FIG. 1. For this purpose, the scan data SD, as were generated in the previously described method Z are received in Step K, and control commands SB are determined or identified from the scan data SD in Step H. In Step J follow-up control commands FSB are then derived on the basis of these control commands SB. This in particular means that the control commands SB can be adopted without modification as follow-up control commands, but also that the control commands SB can be reused with modification. The follow-up control commands FSB are therefore either the control commands SB themselves or modified control commands derived from the control commands SB.

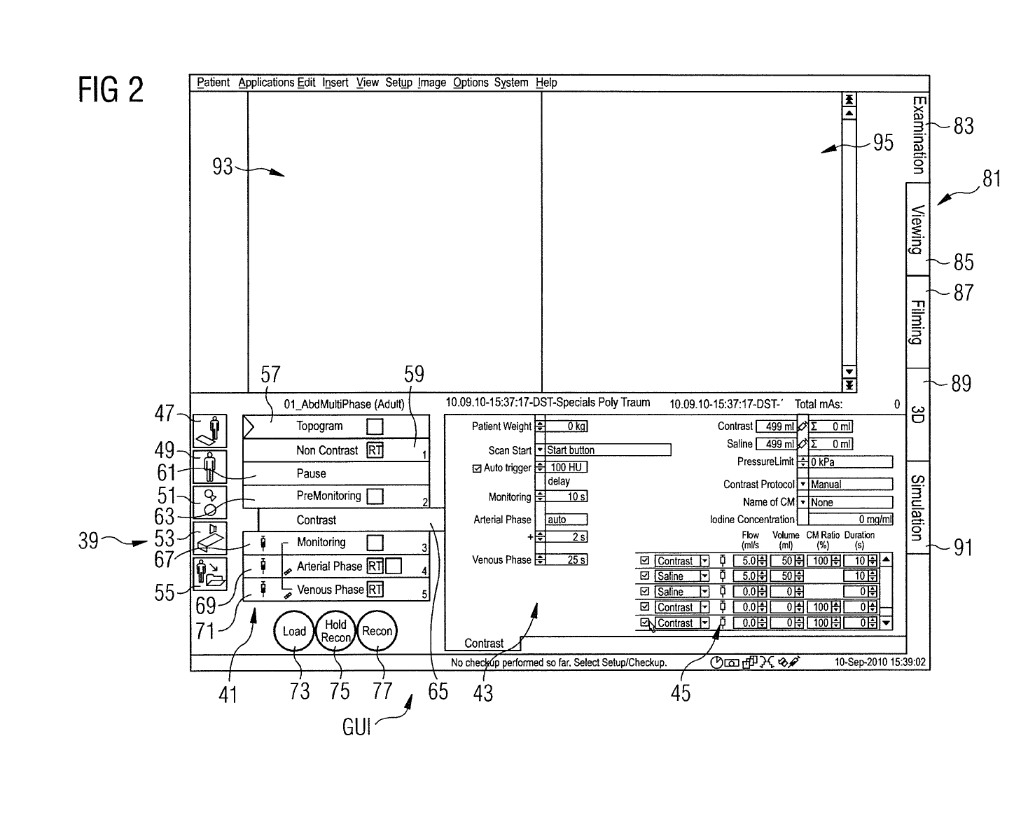

FIG. 2 shows a user interface GUI in a first user mode in which control commands SB can be input to control an imaging system (here and in the following a CT). Depending on a selected imaging system, different control commands SB are respectively required, such that the presentation mode chosen here can only be considered a representative of the entirety of all presentation modes. The control commands SB that are mentioned here likewise represent a selection of possible control commands SB. Nevertheless, every individual input of a control command SB that is mentioned here can yield particular advantages within the scope of the invention.

The user interface GUI is realized as a graphical user interface in connection with an input mouse and a keyboard. It can be presented on the display of a computer, in particular a computer of the imaging system.

The user interface GUI has multiple input and presentation regions 39, 41, 43, 81, 93, 95. Image presentation regions 93, 95 placed in the upper image half of the user interface GUI serve for the presentation of image reproductions that are derived from the image data BD. In the present case of a scan plan for the imaging system, planning images and/or planning symbols can be shown in the image presentation regions 93, 95, which however is not the case in the planning step shown here.

On the right hand side, five tabs 83, 85, 87, 89, 91 that act as index card tabs are arranged in a switching region 81. When they are clicked on, a separate presentation of the user interface GUI that is geared toward a defined planning or, respectively, operating mode respectively appears in the remaining image region of the GUI: a first tab 83 with the title “Examination” serves for the input and viewing of control data SD for examination planning, meaning the planning of the implementation of a tomography scan; a second tab 85 with the title “Viewing” serves to control the image preparation for a user; a third tab 87 with the title “Filming” serves for the control of an output of images via an x-ray film printer. The fourth tab 89 with the title 3D serves for the post-processing of the image data BD, in particular the navigation in three-dimensional data sets that are based on the image data BD. The fifth tab is reserved for developers and serves essentially for programming. In the following (i.e. in FIGS. 2 through 6) only a presentation based on an activation of the first tab 83 is explained in detail.

Five control panels 47, 49, 51, 53, 55 are apparent on the left lower side in a patient input region 39. These serve to create inputs with regard to the examination subject (in particular a patient) to be scanned. Upon activation of the first (i.e. uppermost) control panel 47, an input mask is revealed for the input of patient information such as name, age and much more. The activation of the second control panel 49 situated below this serves for the input of the examination type, for example a body region to be examined. Upon activation of the third control panel 51 that is arranged below the second control panel 49, rough radiation information (high/low) can be entered. Arranged below the third control panel 51 is a fourth control panel 53 that serves for an activation of automated patient commands during a tomography scan, for instance the generation of signals that indicate to the patient when he should hold his breath. The lowermost fifth control panel 55 serves to store the data inputs that were made in the upper four control panels 47, 49, 51, 53.

Arranged to the right of the patient input region 39 is a scan protocol region 41. Switching elements can be inserted and removed as directed by the user. In the present case, eight switching elements 57, 59, 61, 63, 65, 67, 69, 71 have been inserted by a user. Each of the switching elements 57, 59, 61, 63, 65, 67, 69, 71 represents a defined phase during the tomography scan. The individual switching elements 57, 59, 61, 63, 65, 67, 69, 71 are briefly characterized from top to bottom as:

The first switching element 57 (“Topogram”) serves to activate a topogram function, meaning that—in the present case—an overview scan (also called a prescan or topogram) should initially be implemented. The second switching element 59 (“Non-Contrast”) characterizes that the overview scan should take place without administration of contrast to the patient. The third switching element 61 (“Pause”) indicates that a pause is provided after the overview scan in order to evaluate said overview scan. The fourth switching element 63 (“Pre-Monitoring”) stands for measures being initiated at the patient before a detail scan (or primary scan). These measures are specified in detail with the fifth switching element 65 (“Contrast”), which namely indicates that a contrast agent administration is provided. The sixth switching element 67 (“Monitoring”) represents the detail scan in which it is shown by means of the seventh and eighth switching element 69, 71 (“Arterial Phase”, “Venous Phase”) that this detail scan is divided into an arterial phase and a venous phase. Each of the switching elements 57, 59, 61, 63, 65, 67, 69, 71 therefore represents a work step during the total workflow of the tomography scan; additional control commands SB can therefore be input or, respectively, queried at each of these work steps.

These control commands SB are entered in a control command input region 43 whose appearance and whose input regions change depending on the activated switching element 57, 59, 61, 63, 65, 67, 69, 71. In FIG. 2 the switching element 65 “Contrast” is activated, such that control command input fields 45 for contrast agent administration are shown in the control command input region 43: the patient weight can be input in a left column of the control command input region 43 (from top to bottom). Below this, control commands SB are input with regard to how the detail scan should be started—here by pressing a start button—and whether the “Auto-Trigger” function of the imaging system should be activated. “Auto-Trigger” means that the detail scan is begun upon attainment of a certain threshold of contrast agent in the tissue. This threshold is indicated here with 100 HU (Hounsfield Units). Below this, the time duration of the delay of the scan (here 10 seconds) can be set. It is additionally established that, according to an automatic logic, the scan in the arterial phase should be an additional 2 seconds longer; the scan in the venous phase should be 25 seconds long.

In the right column it is initially specified how much contrast agent (“Contrast”) and how much diluted saline are prepared. Below this (“Pressure Limit”), the pressure with which these fluids are administered to the patient can be set. A contrast protocol (“Contrast Protocol”) is created manually; the name of the contrast agent (“Name of CM”) is not specified. Moreover, the iodine concentration (“Iodine Concentration”) can be set manually.

The exact chronological progression of the administration of contrast agent and saline is established in a table under the right column. Only the upper two table lines are relevant here since the others are not used (yet) in the present planning. In the left column it can be indicated what agent—contrast agent (“Contrast”) or saline

(“Saline”)—is specified in detail in the following columns. Next to this, the agent flow is indicated in ml per second (5.0 ml/s in both cases), and in addition to this the volume dose is indicated in ml (here 50 ml of both agents is administered). Indicated next to this is the percentile proportion of contrast imaging agent in the respective agent administration—the contrast agent is 100% of this, the saline is none. Both agents are respectively administered for 10 seconds (Column “Duration”).

Three more control buttons 73, 75, 77 are arranged below the scan protocol region 41. The left control button 73 serves to load the input control commands SB into the imaging system for additional processing; the right control button 77 serves to start a parameter calculation for final control based on the control commands, and the middle control button 75 interrupts such a parameter calculation.

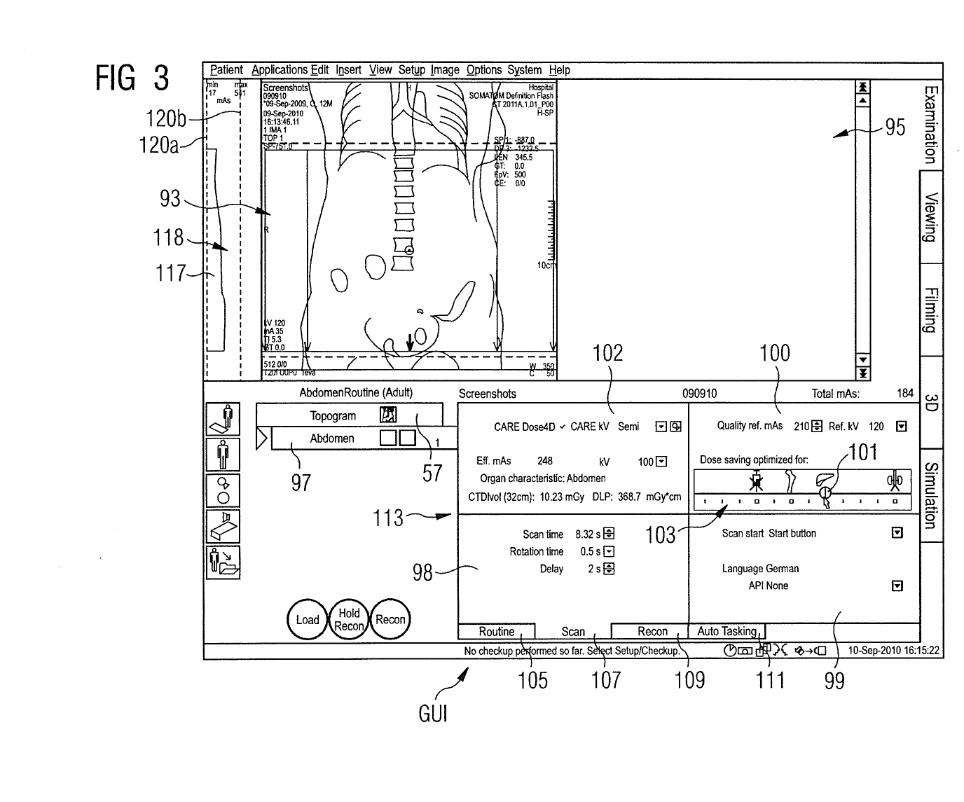

FIG. 3 shows the same user interface GUI, but with fewer selected switching elements 57, 87 in the scan protocol region 41. While the second through eighth switching elements 59, 61, 63, 65, 67, 69, 71 were omitted in this mode, a ninth switching element 97 is added that represents the implementation of a detail scan of the abdomen. The overview scan has already been implemented; in the first image presentation region 93 an image is accordingly indicated, namely the topogram image resulting from this in addition to a dose indication representation 117 (arranged to the left of the topogram image). Here it is shown how much radiation is introduced into the patient (integrated over the width of said patient) when a detail scan is implemented. A scale 118 includes a first threshold line 120a and a second threshold line 120b that represent a lower and an upper dose threshold. In order to be able to implement a reasonable imaging, the lower threshold according to the first threshold line 120a must be exceeded; exceeding the second threshold line 120b should optimally be avoided to prevent excessively high radiation exposures.

A different type of control command input region 113 has been called via the activation of the ninth switching element 97. At the lower end it is apparent that it represents the second tab 107 of four tables 105, 107, 109, 111. An activation of the first (left) tab 105 serves for the input of planning pre-steps, for instance the input of a more precise examination type, for example an abdominal scan of an adult. The second tab 107 further to the right serves for the actual scan planning of the detail scan; the third tab 109 that follows this to the right serves for the planning of the image reconstructions after implementation of the detail scan; and the rightmost, fourth tab serves for the determination of transfer nodes to which the results of the tomography scan should be transmitted. Such a node can be a patient archiving system, for example.

The control command input region 113 shown here has four input rectangles 98, 99, 100, 102. In the upper left input rectangle 102 the program CAREDose4D can be activated or, respectively, deactivated to the above left, which program—just like the program Care kV that can be activated, partially activated or deactivated next to this—serves to automatically implement intelligent dose savings. In the present case, CAREDose4D is activated and Care kV is partially activated. Resulting from these activations are—specified below these—the advance dose specifications of an effective radiation exposure of 248 mAs given a set 100 kV of the radiation source. Specified one row further below this are what is known as the CT dose index and the dose length protocol (likewise automatically calculated).

Additional dose pre-settings can be selected by a user in the upper right input rectangle 100. At the upper left the user indicates what image quality level the user wants to achieve. This is done by the user specifying a normal radiation dose as a reference value (“Quality ref. mAs”) on the basis of which the dose savings programs then derive an image quality to be achieved. This is indicated here with 210 mAs. A reference value for the power of the radiation source (“Ref. kV”) can likewise be specified, here 120 kV. Below these reference specifications, a selection in a scale range 103 for which the radiation dose should be optimized can be made by means of a slide controller 101. The left symbol with the crossed-out syringe indicates that no contrast agent scan should be implemented; the symbol of a bone situated further to the right indicates that an optimization for a bone scan should be made; the following symbol (which represents a liver) serves for the optimization of the dose for a soft tissue scan. The symbol to the far right represents a heart/lung scan for which the dose should be optimized. The slide controller 101 is set here below the liver symbol.

The lower left input rectangle 98 serves to set control commands SB in the form of time parameter values: here the total duration of the detail scan (“Scan Time”) can be fixed (here set at 8.32 seconds), just like the duration of a revolution (“Rotation [sic] Time”) of the detector or the radiation source of the imaging system (here 0.5 seconds). Furthermore, a delay duration (“Delay”) can be specified (2 seconds here) as of which an imaging acquisition should be started after the start of the revolution.

The lower right input rectangle 99 serves to specify more general control commands in the form of user-specific information. Here it is noted how the detail scan is started (namely by pressing a start button); in what language (German here) specifications of the user should be made; and whether a programming interface (“API” —Application Programming Interface) is activated. This is not the case here.

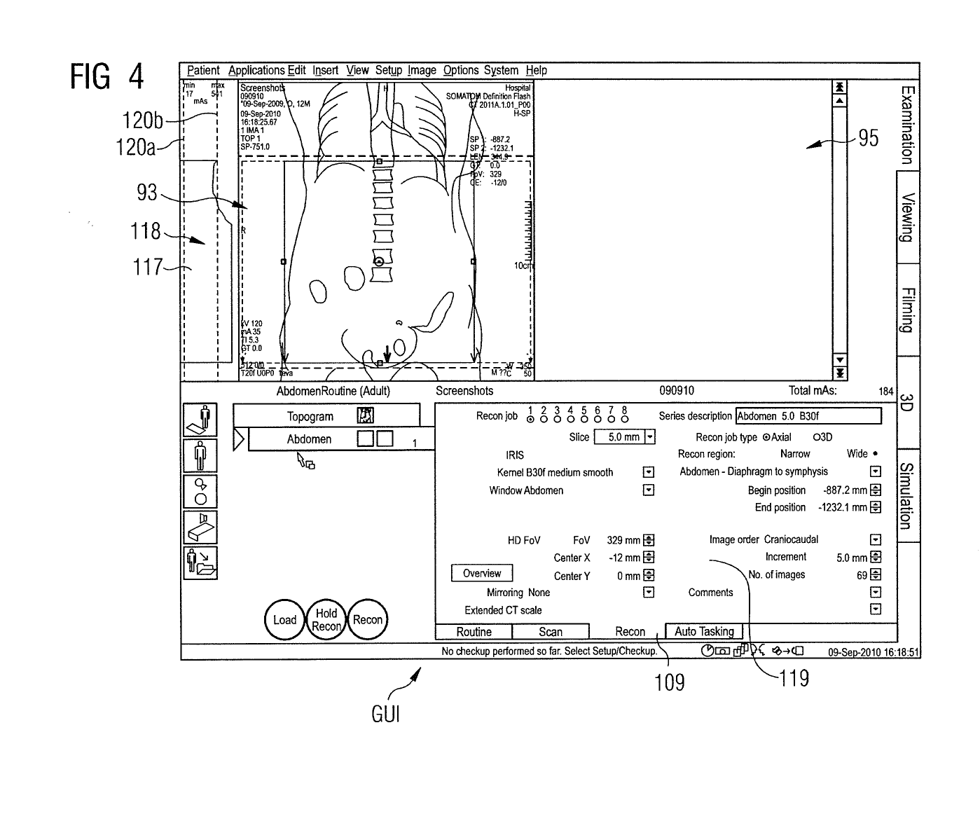

In FIG. 4 the user interface GUI is shown in an additional programming mode, namely after the detail scan planned in FIG. 3. The third tab 109 is therefore now activated so that a new control command input region 119 is activated in turn. Control commands SB for image preparation can be input in this region. These are not discussed in detail; they essentially include information regarding the rendering of slices, known as Field of View, i.e. the region of the patient that should be displayed in the image and measurements corresponding to this. FIG. 4 serves to demonstrate that a significantly greater amount of information is also included in the control commands SB for image preparation than has previously been readable in the DICOM header of a medical technology image, or can be learned from a DICOM Structured Report. Moreover, in the upper left region of the dose specification representation 117, FIG. 4 also shows that a dose overrun beyond the second threshold has occurred in regions. The graphical rendering of the local dose is therefore colored in yellow in the region in which the second threshold line 120b is exceeded to the right, which is only not perceptible here due to the black-and-white drawing. The yellow coloration serves as a warning indicator to the user and can serve as an additional information that can also be stored with the control commands. This can take place in that a snapshot of the entire screen shown here is generated and is linked with the image data BD in the data set DS.

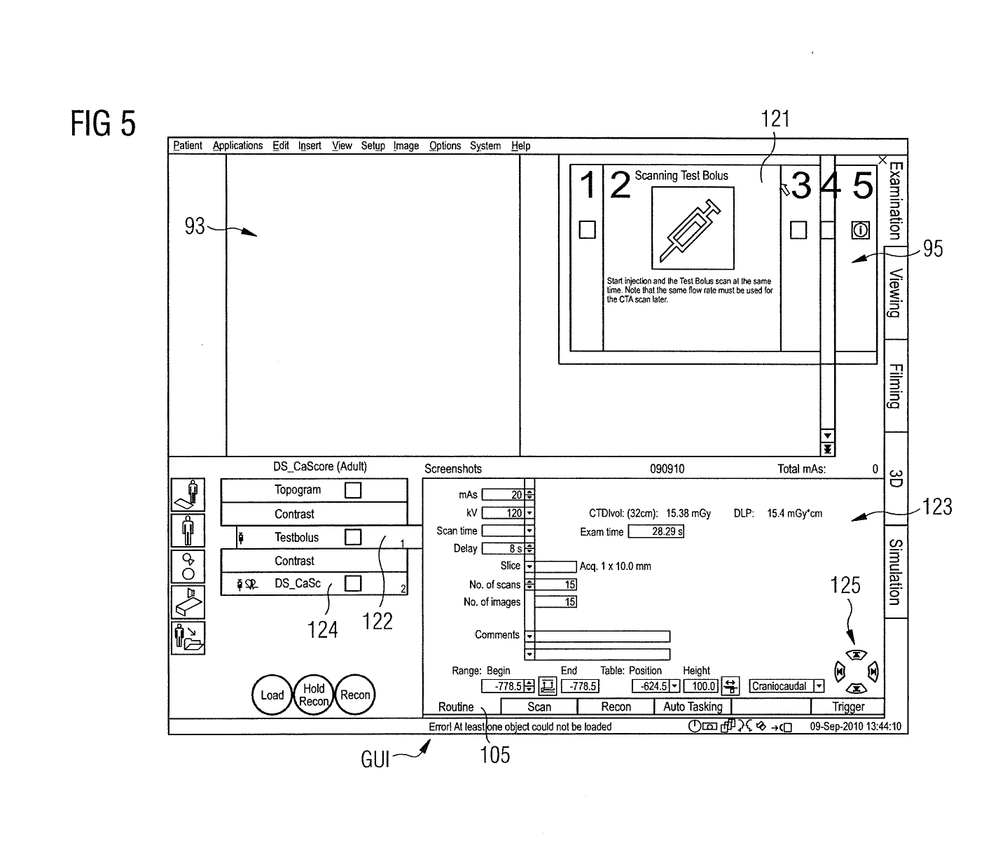

FIG. 5 shows the user interface GUI in an input mode in which two additional switching elements—a tenth switching element 122 and an eleventh switching element 124—have in turn been inserted by a user. In the following, for the sake of clarity only the function of the tenth switching element 122 (which is activated) is discussed, and due to this a control command input region 123 is activated. Connected with this, an additional user guide 121 is arranged in the upper right image half of the user interface GUI (namely in the second image presentation region 95). The user guide 121 has five panes that can be activated in succession, wherein a new input region respectively opens with nine respective input functions for control commands SB. A second pane is activated in the second presentation. The ninth switching element 122 represents the planning of the implementation of a test bolus, i.e. a predefinition of the duration and effect of a contrast agent injection into a patient. Contrast agent administrations can consequently be generated with targeted precision due to the measurement results generated by the test bolus. In addition to symbols, the panes of the user guide 121 also include instructions to the user who, based on these, can implement the planning of steps of the test bolus on the basis of control command inputs. The user can thereby keep the specifications from the instructions or can also deviate from these in a targeted manner. This is in turn documented by a corresponding storage of the scan data. In addition to numerical inputs, position inputs can also be implemented here in a position control region 125. They pertain here to the positioning of a patient and the slice image acquisitions to be implemented during the test bolus. Like all other non-numerical control commands, these position inputs can be generated both as a graphical representation (i.e. are stored as an image) and as a number sequence (for instance as numerical information that is generated in the background).

In particular, it is necessary to document when the user deviates from the instructions from the plates of the user guide 121. Very generally, it is preferred to document in the scan data SD every deviation from specifications that are already preset in the protocols.

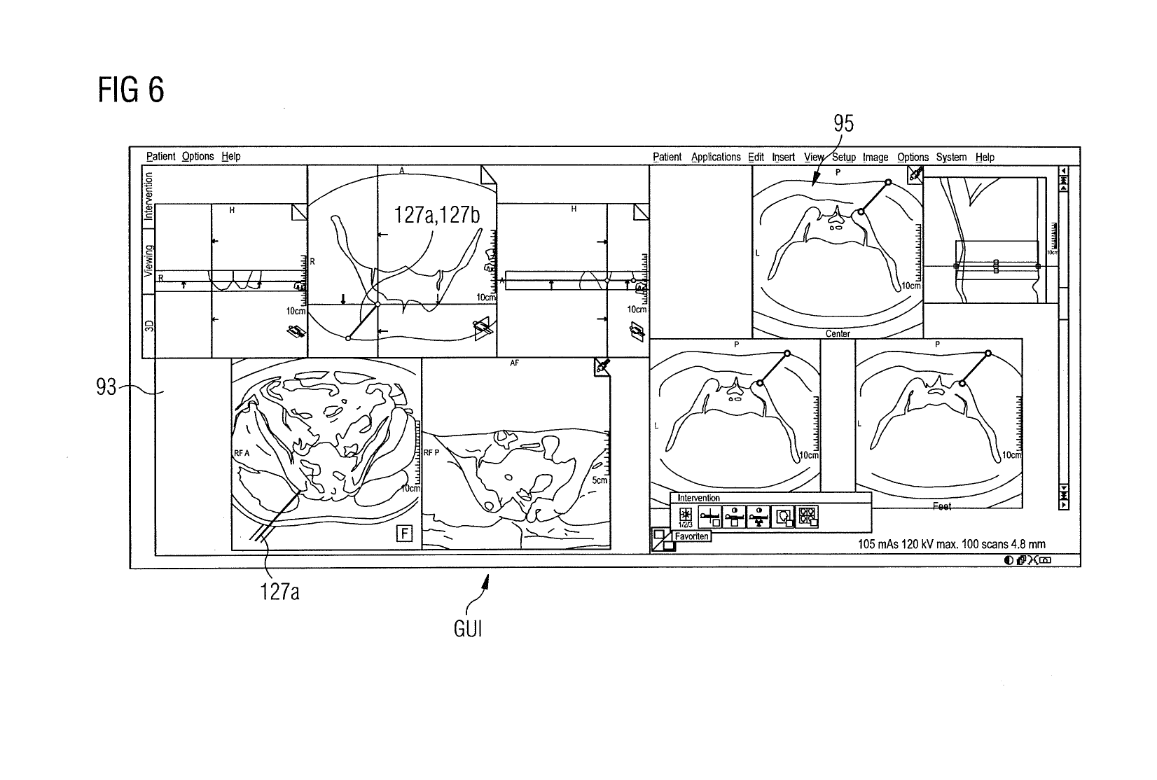

FIG. 6 shows a detail of the user interface GUI, namely the two image presentation regions 93, 95. Within the scope of this exemplary embodiment, image data BD accompanying a minimally invasive procedure are acquired by means of a needle 127a in what is known as a fluoroscopy or biopsy. In a fluoroscopy, the imaging scan is implemented continuously during the minimally invasive procedure, while in a biopsy scans are initiated and implemented (sequentially) again in a biopsy after activated by a user.

The needle 127a and the corresponding procedure or, respectively, its planning here are documented only with images in the first image presentation region 93, while the second image presentation region 95 serves to document a needle detection logic of the imaging system. A needle path 12b as it was planned before the beginning of the procedure on the basis of the previously acquired image data BD from prescans is documented in the upper middle image. At the same time, the needle 127a is also shown whose position precisely coincides (i.e. is congruent) with the planned path 127b in the present case. The lower image shows a three-dimensional reconstruction as it was calculated after implementing scans. Here only the needle 127a is visible. With the use of the images shown here, it can be documented whether the minimally invasive procedure and the path planning were executed correctly. Deviations from the planned path are likewise documented as a possibly incorrect path plan.

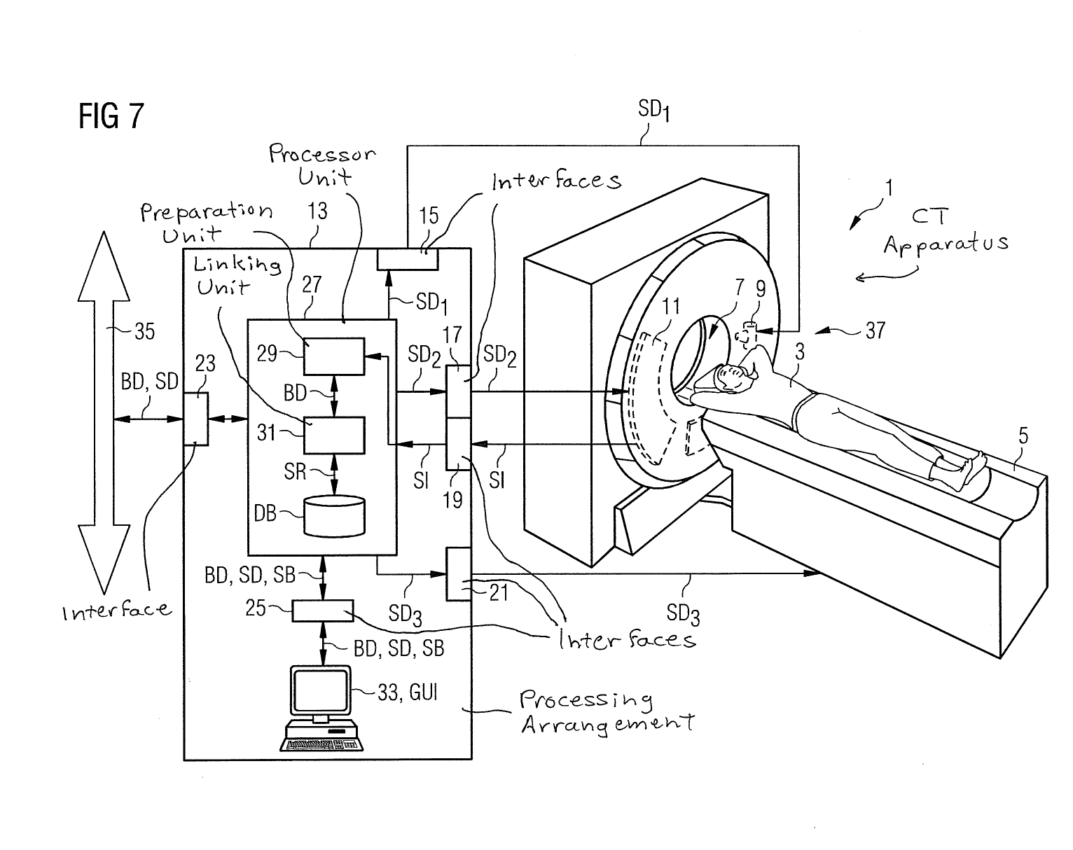

FIG. 7 shows an exemplary embodiment of a medical imaging technology system 1 according to the invention, realized here as a CT apparatus 1 has a scanner (CT data acquisition unit) 37 and a data processing arrangement 13. The scanner 37 has an x-ray source 9 that, rotating along a gantry together with a detector arrangement 11 that is situated opposite the x-ray source 9 on the other side of the rotation axis, is arranged around a patient opening 7. An examination subject 3—here a patient 3—can be driven into the patient opening 7 on a displaceable subject support table 5.

The data processing arrangement 13 is essentially realized as a processor unit 27 that interacts with various interfaces 15, 17, 19, 21, 23, 25. It is connected with an input computer 33 via a first (input/output) interface 25. This input computer 33 serves as the user interface GUI. The processor unit 27 sends first control data SD₁ to the scanner unit 37 via a second (output) interface 15 to control the x-ray source 9. The processor unit 27 sends second control data SD₂ to the detector arrangement 11 via a third (output) interface 17. Via a fourth (input) interface 19 it receives signals SI from the detector arrangement 11. Third control data SD₃ are sent to the subject bearing table 5 via a fifth (output) interface 21, on the basis of which third control data SD₃ the attitude of the examination subject 3 can be adapted. The processor unit 27 communicated with a patient archiving system 35 via a sixth (input/output) interface 23. For example, the patient archiving system 35 is set up decentrally at a central computer of a clinic and can communicate with multiple imaging systems such as the imaging system 1 present here or imaging systems of other design.

A preparation unit 29 that, during operation, prepares image data that are derived from the signals SI from a scan process of the scanner arrangement 37 is arranged in the processor unit 27. The preparation unit 29 communicates with a linking unit 31 that links these image data BD with a data set DS. This data set DS represents the user interface GUI during a reception of control commands SB by a user (not shown). To select representative control commands RSB, the linking unit 31 follows selection rules SR from a database DB that (in the present case) is arranged within the data processing arrangement 13 (but can also be externally linked with it via an interface).

The image data BD, the control commands SB and the scan data SD are communicated between the processor unit 27 and the computer 33 via the first interface 25. The image data BD and the scan data SD are likewise relayed or, respectively, can in turn be referred from this to the patient archiving system 35 via the sixth user interface 23. For example, a reference (to the scan data SD in particular) from the patient archiving system 35 can serve to extract control commands SB included in the scan data SD for a follow-up scan by the imaging system 1, and to derive from these control commands SB follow-up control commands FSB as are explained in detail in the context of FIG. 1.

Although modifications and changes may be suggested by those skilled in the art, it is the intention of the inventor to embody within the patent warranted hereon all changes and modifications as reasonably and properly come within the scope of his contribution to the art.

I claim as my invention:

1. A method to generate scan data with a medical imaging system, comprising:
via a user interface of a computer, entering control commands into the computer that control an imaging scan process by, or an image output from, a medical imaging system operated by the computer;
implementing said imaging scan process with said medical imaging system controlled by said computer to produce image data; and
from said computer, storing said image data generated by said imaging scan process in a memory linked with a data set representing said user interface during reception of said image data, said image data linked with said data set comprising a stored set of scan data.

2. A method as claimed in claim 1 comprising generating said data set as images representing said user interface during said reception of said image data.

3. A method as claimed in claim 2 wherein said images respectively comprise snapshots of a graphical user interface of said user interface.

4. A method as claimed in claim 2 wherein said images represent an imaging workflow sequence of said imaging scan process.

5. A method as claimed in claim 1 comprising generating said data set as machine-readable data.

6. A method as claimed in claim 6 comprising generating said data set to comprise both images and machine-readable data.

7. A method as claimed in claim 1 comprising storing said scan data in said memory with said image data and said data set inseparably linked for retrieval of scan data from said memory.

8. A method as claimed in claim 1 comprising, in said computer, generating said data set automatically based on a predetermined selection rule accessible by said computer.

9. A method as claimed in claim 8 comprising employing, as said selection rule, a rule that includes at least one relevance criterion that relates to relevance of individual control commands or groups of control commands with respect to an effect thereof on said image data or on said imaging scan process.

10. A method as claimed in claim 1 comprising selecting content of said data set at least partially by an entry by a user made via said user interface.

11. A method as claimed in claim 1 comprising implementing said imaging scan process as part of an invasive procedure in a body of a subject, and generating said data set to comprise data representing at least one of planning or a prediction of said invasive procedure.

12. A method to generate follow-up control commands for an imaging scan process with a medical imaging system, comprising:
via a user interface of a computer, entering control commands into the computer that control an imaging scan process by, or an image output from, a medical imaging system operated by the computer;
implementing said imaging scan process with said medical imaging system controlled by said computer to produce image data;
from said computer, storing said image data generated by said imaging scan process in a memory linked with a data set representing said user interface during reception of said image data, said image data linked with said data set comprising a stored set of scan data;
retrieving said scanned data from said memory and, in said computer, identifying at least one of said control commands from said data set of the retrieved scan data; and
in said computer, deriving at least one follow-up control command from the identified control command in said data set of the retrieved scan data.

13. A medical imaging system comprising:
a medical data acquisition unit configured to interact with a subject to acquire medical data therefrom;
a control unit configured to operate said medical data acquisition unit;
a user interface in communication with said control unit, said control unit being configured to receive control commands entered into the control unit via the user interface that control an imaging scan process by, or an image output from, said medical data acquisition unit;
said control unit being configured to implement said imaging scan process with said medical data acquisition unit to produce image data; and
said control unit being configured to store said image data generated by said imaging scan process in a memory linked with a data set representing said user interface during reception of said image data, said image data linked with said data set comprising a stored set of scan data.

14. A medical imaging system comprising:
a medical data acquisition unit configured to interact with a subject to acquire medical data therefrom;
a control unit configured to operate said medical data acquisition unit;
a user interface in communication with said control unit, said control unit being configured to receive control commands entered into the control unit via the user interface that control an imaging scan process by, or an image output from, said medical data acquisition unit;
said control unit being configured to implement said imaging scan process with said medical data acquisition unit to produce image data; and
said control unit being configured to store said image data generated by said imaging scan process in a memory linked with a data set representing said user interface during reception of said image data, said image data linked with said data set comprising a stored set of scan data;
said control unit being configured to retrieve said scanned data from said memory and to identify at least one of said control commands from said data set of the retrieved scan data; and
said control unit being configured to derive at least one follow-up control command from the identified control command in said data set of the retrieved scan data.

15. A non-transitory, computer-readable data storage medium encoded with programming instructions, said data storage medium being loaded into a computerized control unit of a medical imaging system and said programming instructions causing said computerized control unit to:
via a user interface, receive control commands into the control unit that control an imaging scan process by, or an image output from, a medical imaging system operated by the computer;
implement said imaging scan process with said medical imaging system controlled by said computer to produce image data; and
store said image data generated by said imaging scan process in a memory linked with a data set representing said user interface during reception of said image data, said image data linked with said data set comprising a stored set of scan data.

16. A non-transitory, computer-readable data storage medium encoded with programming instructions, said data storage medium being loaded into a computerized control unit of a medical imaging system and said programming instructions causing said computerized control unit to:
via a user interface of a computer, receive control commands into the control unit that control an imaging scan process by, or an image output from, a medical imaging system operated by the computer;
implement said imaging scan process with said medical imaging system controlled by said computer to produce image data;
store said image data generated by said imaging scan process in a memory linked with a data set representing said user interface during reception of said image data, said image data linked with said data set comprising a stored set of scan data;
retrieve said scanned data from said memory and, in said computer, identifying at least one of said control commands from said data set of the retrieved scan data; and
derive at least one follow-up control command from the identified control command in said data set of the retrieved scan data.
